(19) United States
(12) Patent Application Publication
(10) Pub. No.: US 2014/0221829 A1
(43) Pub. Date: Aug. 7, 2014

(54) LOCALIZED FLUORESCENCE EXCITATION IN WHOLE BODY OPTICAL IMAGING

(71) Applicant: THE TEXAS A&M UNIVERSITY SYSTEM, COLLEGE STATION, TX (US)

(72) Inventors: Kristen Carlson Maitland, College Station, TX (US); Jeffrey D. Cirillo, College Station, TX (US); Ying Kong, Germantown, TN (US); Joel N. Bixler, Albuquerque, NM (US)

(73) Assignee: THE TEXAS A&M UNIVERSITY SYSTEM, COLLEGE STATION, TX (US)

(21) Appl. No.: 14/171,367

(22) Filed: Feb. 3, 2014

Related U.S. Application Data
(60) Provisional application No. 61/759,866, filed on Feb. 1, 2013.

(51) Int. Cl. A61B 5/00

(52) U.S. Cl. CPC A61B 5/0071; A61B 5/0084
USPC 600/431; 600/478

(57) ABSTRACT

Methods and systems for whole body optical imaging using localized fluorescence excitation.

### CROSS-REFERENCE TO RELATED APPLICATION

This application claims the benefit of U.S. Provisional Application No. 61/759,866, filed Feb. 1, 2013, which application is expressly incorporated herein by reference.

### BACKGROUND OF THE INVENTION

Respiratory diseases are the most common cause of death in humans worldwide. The World Health Organization currently estimates that roughly one-third of the world's population is infected with tuberculosis (TB), caused by the Mycobacterium tuberculosis (Mtb). In the year 2011 alone, 8.7 million people fell ill with TB and another 1.4 million died. While the risk of developing symptoms from the latent condition is only 10%, this number increases greatly if the individual is also infected with an immune compromising disease such as HIV. TB is a treatable and curable disease, typically combated with a six-month course of antimicrobial drugs, and the use of these treatments has significantly decreased the mortality rate for TB over the last quarter century. Despite this, multi-drug resistant TB strains generate concern among medical experts and demand the need for the development of new antimicrobial therapies. In addition to this, the current vaccine, Mycobacterium bovis Bacillus Calmette Guerin (BCG), displays variable efficacy (0-80%) depending on the population group being vaccinated. Currently, researchers typically rely on animal studies to help assess the effectiveness of new therapeutic agents. These studies employ sacrifice at discrete time points, tissue homogenization, and colony growth. These factors combine to greatly limit temporal and spatial resolution of the bacteria in tissue. Thus, the development of an experimental technique that could provide rapid feedback regarding the efficacy of a therapeutic agent in an animal model of a respiratory infection could greatly benefit the development of such vaccines.

Optical sensing provides a promising solution to the challenge of studying bacterial infection dynamics inside an animal model over time. Whole-animal optical imaging has significantly advanced the ability to detect and monitor disease progression at a cellular and molecular level. Such systems are still inherently limited by tissue scattering and absorption, and thus, the study of physiologically relevant infections is often impossible. Because of this, improving the threshold of detection for a whole-animal imaging system could ultimately allow for the study of early stage infection physiology and development of therapeutic treatments.

Despite the development of optical sensing systems noted above, there exists a need for improved illumination in whole body imaging systems and methods. The present invention seeks to fulfill this need and provides further related advantages.

### SUMMARY OF THE INVENTION

The present invention provides systems and methods for whole body optical imaging using localized fluorescence excitation.

In one aspect, the invention provides a method for detecting fluorescence from a body. In one embodiment, the method comprises:

(a) introducing a light source into a body;

(b) illuminating a portion of the body with the light source, wherein the light source emits light having a wavelength and intensity sufficient to elicit fluorescence from at least a portion of the body illuminated; and

(c) detecting the fluorescence from outside the body.

In certain embodiments, introducing the light source into the body comprises introducing the light source through an external orifice of the body. In other embodiments, introducing the light source into the body comprises introducing the light source through an incision.

In certain embodiments, detecting the fluorescence comprises whole body imaging.

In another aspect of the invention, a method for monitoring the progression of a disease or condition is provided. In one embodiment, the method comprises:

(a) introducing a light source into a body of a subject;

(b) illuminating a portion of the body with the light source, wherein the light source emits light having a wavelength and intensity sufficient to elicit fluorescence from at least a portion of the body illuminated, and wherein the portion of the body illuminated comprises a fluorescent substance indicative of the state of a disease or condition;

(c) detecting the fluorescence from outside the body to provide a first fluorescent measurement;

(d) waiting a pre-determined period of time;

(e) illuminating the portion of the body as in step (b);

(f) detecting the fluorescence from outside the body to provide a second fluorescent measurement; and

(g) comparing the first fluorescent measurement and the second fluorescent measurement to determine the progression of the disease or condition.

In certain embodiments, the steps (d)-(g) are repeated one or more times to create a profile.

In certain embodiments, the fluorescent substance is a cell. In other embodiments, the fluorescent substance is a pathogen.

In a further aspect, the invention provides a method for monitoring the effectiveness of a therapeutic agent. In one embodiment, the method comprises:

(a) introducing a light source into a body of a subject;

(b) illuminating a portion of the body with the light source, wherein the light source emits light having a wavelength and intensity sufficient to elicit fluorescence from at least a portion of the body illuminated, and wherein the portion of the body illuminated comprises a fluorescent substance indicative of the state of a disease or condition;

(c) detecting the fluorescence from outside the body to provide a first fluorescent measurement;

(d) administering a therapeutic agent to the subject;

(e) illuminating the portion of the body as in step (b);

(f) detecting the fluorescence from outside the body to provide a second fluorescent measurement; and

(g) comparing the first fluorescent measurement and the second fluorescent measurement to determine the effectiveness of the therapeutic agent.

In certain embodiments, steps (e)-(g) are repeated one or more time to provide a profile.

In other embodiments, steps (d)-(g) are repeated one or more times to provide a profile.

In certain embodiments, the fluorescent substance is a cell. In other embodiments, the fluorescent substance is a pathogen.

In other aspects, systems for performing the methods of the invention are provided.

### DESCRIPTION OF THE DRAWINGS

The foregoing aspects and many of the attendant advantages of this invention will become more readily appreciated as the same become better understood by reference to the following detailed description, when taken in conjunction with the accompanying drawings.

FIG. 1 is a schematic illustration of a representative fluorescent microendoscope used for in vivo microendoscopic imaging and for localized internal fluorescence excitation for whole animal imaging. LED (light emitting diode), CL (collimating lens), EX (excitation filter), DM (dichroic minor), OL (objective lens), EM (emission filter), FL (focusing lens), CCD (charged coupled device) camera.

FIGS. 2A-2F compare macroscopic IVIS images collected with fiber bundle excitation in the mouse lung for 10¹, 10², 10³, 10⁴, and 10⁵ CFU tdTomato expressing BCG (2A-2E, respectively) and 10⁵ CFU BCG with vector backbone (2F). Scale bar units are in (photons/sec/cm²/sr)/(μW/cm²).

FIGS. 3A-3C compare macroscopic IVIS images collected using conventional epi-illumination from the IVIS system for infections of 10⁴ and 10⁵ CFU tdTomato expressing BCG (3A and 3B, respectively) and 10⁵ CFU BCG with vector backbone (3C). Scale bar units are in (photons/sec/cm²/sr)/(μW/cm²).

FIG. 4 compares measured fluorescence intensity using IVIS image collection and Living Image software with localized fluorescence excitation through a fiber bundle with LED source. Inoculation dose of tdTomato expressing BCG bacteria is indicated from 10⁵ to 10¹ CFU as compared to a normal control of BCG bacteria with vector backbone.

### DETAILED DESCRIPTION OF THE INVENTION

The present invention provides systems and methods for whole body optical imaging using localized fluorescence excitation.

In one aspect, the invention provides a method for detecting fluorescence from a body of a subject. In one embodiment, the method comprises:

(a) introducing a light source into a body of a subject;

(b) illuminating a portion of the body with the light source, wherein the light source emits light having a wavelength and intensity sufficient to elicit fluorescence from at least a portion of the body illuminated; and

(c) detecting the fluorescence from outside the body of the subject.

In the methods of the invention, fluorescence is elicited by illuminating a portion of a body of a subject with light emitted from a light source. In the methods, fluorescence is elicited by localized excitation. As used herein, the term “localized excitation” refers to excitation or illumination of a portion of the body by internal excitation. In the methods, the light source or light guide that conducts excitation light from the light source is introduced into the body and positioned to effect the desired localized excitation.

To effect efficient localized excitation, the light source is introduced into the subject's body (i.e., internal excitation). In certain embodiments, introducing the light source into the body comprises introducing the light source through an external orifice of the body. Representative orifices include oral, nasal, anal, and vaginal orifices. In other embodiments, introducing the light source into the body comprises introducing the light source through an incision made in the subject's body. In certain embodiments, introducing the light source into the body comprises introducing the light source using a catheter.

In the practice of the methods of the invention, localized excitation is achieved by introducing a light source into the subject's body. Representative light sources suitable for internal excitation include light-emitting diodes (LEDs) and laser diodes (LDs). It will be appreciated that suitable light sources include light sources that are not localized within a body during excitation, but are external to the body and are coupled to a light guide that is localized within the body. The term “light source” therefore includes a light guide that coupled to a light source that conducts excitation light from the light source (external or internal) to the desired target within the body. Representative light sources suitable for coupling to a light guide include light-emitting diodes (LEDs), laser diodes (LDs), lasers, and incoherent light sources. Suitable light guides include those known in the art. Representative light guides include optical fibers and optical fiber bundles. In certain embodiments, the excitation light is transmitted through a light guide.

The wavelength (or wavelength band) of excitation light will depend on the light source and selected based on the nature of fluorescence sought to be elicited. The excitation light can be monochromatic (e.g., LED, LD, or laser; 488 nm, 633 nm) or can be a band of wavelengths determined by the light source and appropriate filters. Excitation light can be from one or more monochromatic sources or wavelength bands. In certain embodiments, the excitation wavelength is one or more wavelengths (or wavelength bands) of visible or near-infrared light.

In certain embodiments of the method, the portion of the body illuminated with light comprises a fluorescent agent introduced into the body as a fluorescent agent or as a latent fluorescent agent. As used herein, the term “fluorescent agent” refers to a fluorescent substance such as a fluorescently-labeled material (e.g., fluorophore conjugate or fluorescent protein) and the term “latent fluorescent agent” refers to a substance that becomes fluorescent after introduction to the body (i.e., a fluorescent agent generated by action within the body such as a fluorogenic product produced by enzymatic action from a non-fluorogenic substrate). In certain embodiments, the fluorescent agent or the latent fluorescent agent is a cell (e.g., cancer cell). In other embodiments, the fluorescent agent or the latent fluorescent agent is a pathogen (e.g., infectious agent such as a microorganism including bacteria or viruses.

In the methods of the invention fluorescence is detected from outside the body. In the methods, detecting the fluorescence outside the body refers to measuring fluorescence externally from the whole body or measuring fluorescence externally from a portion of the whole body. Detecting the fluorescence resulting from localized excitation does not include detecting the fluorescence internal to the body (e.g., detected fluorescence does not include detecting fluorescence directed to fluorescence detector via a light guide). In certain embodiments, detecting the fluorescence comprises whole body imaging.

Fluorescence from outside the body of the subject can be detected by fluorescence detectors known in the art. Representative detectors include photodiodes, photodiode arrays, photomultiplier tubes, CCD cameras, and CMOS cameras.

The methods of the invention described above can be used in animal models for monitoring the progression of a disease or condition or for monitoring the effectiveness of a therapeutic agent.

In one aspect, the invention provides a method for monitoring the progression of a disease or condition in a subject. In one embodiment, the method comprises:

(a) introducing a light source into a body of a subject;

(b) illuminating a portion of the body with the light source, wherein the light source emits light having a wavelength and intensity sufficient to elicit fluorescence from at least a portion of the body illuminated, and wherein the portion of the body illuminated comprises a fluorescent substance indicative of the state of a disease or condition;

(c) detecting the fluorescence to provide a first fluorescent measurement (e.g., a first fluorescent image);

(d) waiting a pre-determined period of time;

(e) illuminating the portion of the body as in step (b);

(f) detecting the fluorescence to provide a second fluorescent measurement (e.g., a second fluorescent image); and

(g) comparing the first fluorescent measurement and the second fluorescent measurement to determine the progression of the disease or condition.

In certain embodiments, the fluorescent substance indicative of the state of a disease or condition is introduced into the subject's body. The fluorescence substance can be a pathogen or a collection of cells (e.g., a tumor). It will be appreciated that the progression can be monitored over time to provide a profile by repeating steps (d)-(g) one or more times.

In another aspect, the invention provides a method for effectiveness of a therapeutic agent in a subject. In one embodiment, the method comprises:

(a) introducing a light source into a body of a subject;

(b) illuminating a portion of the body with the light source, wherein the light source emits light having a wavelength and intensity sufficient to elicit fluorescence from at least a portion of the body illuminated, and wherein the portion of the body illuminated comprises a fluorescent substance indicative of the state of a disease or condition;

(c) detecting the fluorescence to provide a first fluorescent measurement (e.g., a first fluorescent image);

(d) administering an amount of a therapeutic agent to the subject;

(e) optionally waiting a pre-determined period of time;

(f) illuminating the portion of the body as in step (b);

(g) detecting the fluorescence to provide a second fluorescent measurement (e.g., a second fluorescent image); and

(h) comparing the first fluorescent measurement and the second fluorescent measurement to determine the effectiveness of the therapeutic agent.

In certain embodiments, the fluorescent substance indicative of the state of a disease or condition is introduced into the subject's body. The fluorescence substance can be a pathogen or a collection of cells (e.g., a tumor). It will be appreciated that the nature and amount of the therapeutic agent administered can be varied. One or more therapeutic agents can be administered.

It will be appreciated that single dose effectiveness can be monitored over time to provide a profile by repeating steps (e)-(h) one or more times. Alternatively, multiple dose effectiveness can be monitored over time to provide a profile by repeating steps (e)-(h) one or more times.

The following is a description of a representative system and methods of the invention. More specifically, the following is a description of carrying out a representative method of the invention, whole animal imaging using in vivo illumination.

In one embodiment, the present invention provides a fiber-bundle-coupled light source coupled into a Caliper IVIS Lumina II whole-animal optical imaging system, allowing for enhanced excitation of fluorescence from tdTomato expressing BCG in vivo over conventional epi-illumination in the whole animal imaging system. A 535 nm LED was collimated and launched into a 10,000 element fiber bundle with an outer diameter of 0.66 mm. The fiber bundle can be inserted through an intra-tracheal catheter into the lung of a mouse. Fluorescent emission can either be (1) collected by the bundle and imaged onto the surface of a CCD camera for localized detection or (2) imaged by the whole animal imaging system providing macroscopic information. Results from internal localized excitation and external whole body detection indicate the potential to detect and image bacterial infections down to 10-100 colony forming units, an increase in sensitivity over the conventional whole body imaging with epi-illumination by up to 6 orders of magnitude (1,000,000×). The fluorescence excitation technique of the invention for whole animal imaging has the potential to allow for functional studies, enhancing the ability to assess new therapeutic agents.

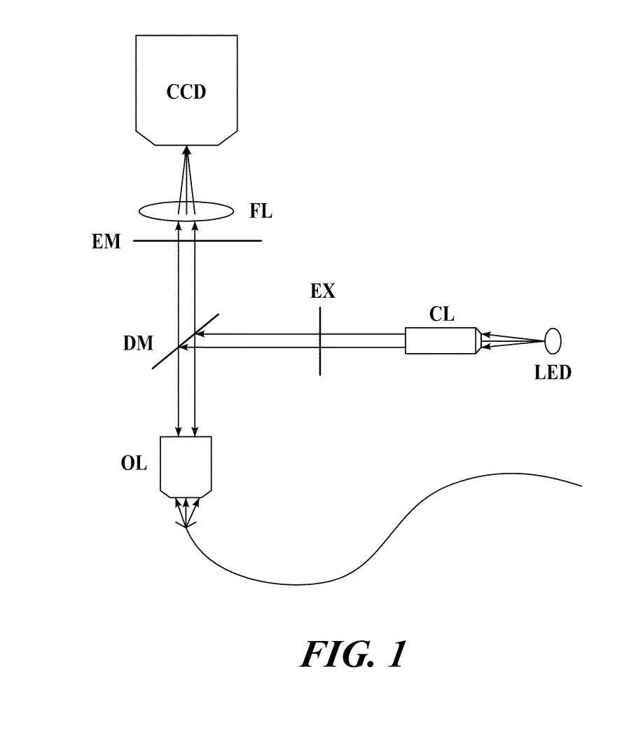

Optical System Design. FIG. 1 illustrates a representative fluorescent microendoscope that was integrated into a whole-animal scanner and used for localized excitation. Excitation and emission wavelengths were selected for the detection of tdTomato fluorescence. tdTomato is a fluorescent protein with a longer excitation wavelength (about 550 nm) than the more common green fluorescent protein (GFP) around 488 nm. The excitation wavelength of the optical system can be easily adjusted to any visible or near infrared wavelength, depending on the targeted fluorophore. In this experiment, a light emitting diode (Thorlabs M530L2) centered at 530 nm with a 31 nm bandwidth is used as the excitation source. The light is collimated and then passes through an excitation filter (Semrock FF01-531/40) and dichroic mirror (Semrock FF562-Di). The light is then launched into a fiber bundle (Sumitomo) with 0.66 mm outer diameter, 450 μm field of view, 10,000 individual fibers, and 3 μm resolution. A fiber bundle was selected over a single optical fiber to enable microendoscopic imaging of the excitation site. This particular fiber bundle was selected due to its ability to fit through an intratracheal catheter. The fiber bundle guides the excitation light to its distal tip, which can be placed directly into the lungs of a mouse through an intratracheal catheter. The resulting fluorescence emission from the sample can be collected by the same fiber bundle, filtered by an emission filter (Chroma HQ572LP) and imaged onto a 1.45 megapixel CCD camera (Qlmaging Exi Blue). This microendoscopic imaging capability can assist in placement of the fiber bundle, by providing feedback on the fluorescence generated near the tip of the fiber bundle. The camera is not required for localized excitation; therefore, a simplified localized excitation optical system could consist of a light source, such as the LED, and a single optical fiber or fiber bundle.

The microendoscope and fiber bundle excitation source was then integrated into an IVIS Lumina II whole-animal imaging system (Perkin Elmer) through an access port on the side of the light-tight enclosure. By integrating the microendoscope with the IVIS system, several features of the combined system can be exploited. First, the IVIS CCD can be used to localize and track the fiber bundle position inside the animal, allowing for proper positioning of the bundle inside an animal prior to data collection. This can be done by blocking the IVIS excitation and setting the emission filter to open. Second, the microendoscope can be used as a localized excitation source for the IVIS system. Excitation is then limited to the tissue localized at or near the tip of the probe. Because the localized excitation light does not need to pass through the skin and thick tissue to the lung, excitation light absorption by tissue and unwanted autofluorescence from tissue are greatly reduced, enhancing excitation efficiency and sensitivity to targeted fluorescence. Thus, by combining these separate excitation and detection systems, light intensity at the desired target location can be achieved. The integration of the fiber bundle imaging system into the whole-animal imager also allows for simultaneous macroscopic and microscopic imaging of infections in vivo.

In Vivo Imaging Animal Model. All animal studies were approved by the Texas A&M Institutional Animal Care and Use Committee. For all imaging experiments, Mycobacterium bovis Bacillus Calmette-Guerin (BCG) was selected for the safety considerations owing to its lack of virulence in humans. BCG bacterial colonies expressing the fluorescent protein, tdTomato, and negative control expressing only the vector backbone where grown on agar as described in Kong, Y., A. R. Akin, K. P. Francis, N. Zhang, T. L. Troy, H. Xie, J. Rao, S. L. Cirillo, and J. D. Cirillo, Whole-body imaging of infection using fluorescence. Curr Protoc Microbiol, 2011. Chapter 2: p. Unit2C 3.

Four mice infected with tdTomato labeled BCG ranging from 10⁷ to 10⁴ colony forming units (CFU) and one mouse infected with a negative control of 10⁷ CFU BCG were imaged using internal excitation. Each mouse was anesthetized using ketamine-xylazine. A catheter was inserted into the trachea of the animal, which was then placed inside the IVIS system. The animal was placed on the stage such that it was lying on its back, and the paws were secured to the stage using tape to ensure the animal did not move during imaging. A 0.66 mm OD fiber bundle was inserted into the catheter, and advanced until fluorescent signal was observed using the CCD camera. The fiber bundle was marked to ensure the insertion distance was similar between animals, and to help guide fiber insertion for the negative control animal. The fiber bundle was also secured to the stage to ensure it did not move during imaging by a piece of electrical tape. Fluorescence images were recorded using the microendoscope CCD. IVIS acquisition was performed with the excitation filter set to “block” and the emission filter set to 580, 600, 620, and 640 nm, as well as “open.” The open position was used for determining the location of the tip of the fiber bundle inside the animal. The exposure time for each wavelength was set to “auto.” For comparison to conventional illumination, each animal was also imaged using traditional IVIS excitation (epi-illumination) and emission. For these sequences, the excitation filter was set to 535 nm, the exposure was set to 10s, and the emission sequence was set to cycle through 580, 600, 620, and 640 nm.

The experiments were then repeated for inoculations ranging from 10⁵ to 10¹ CFU. For each concentration, three animals were imaged. All images were compared to a 10⁵ CFU negative control. The same animal procedure was used for preparing and imaging each animal, and the same settings were used with the IVIS system.

Following imaging, each animal was sacrificed, and the lung tissue was excised. Excised lungs were then placed in a petri dish and imaged inside the IVIS system using its traditional excitation. The three sets of lungs for each CFU were placed in the dish along with the negative control, and images were taken using the same range of emission wavelengths. Lung tissue was then homogenized and bacteria were quantified by colony viability counts following one month of bacterial culture.

Image analysis of the IVIS images was performed using Living Image software provided with the IVIS system.

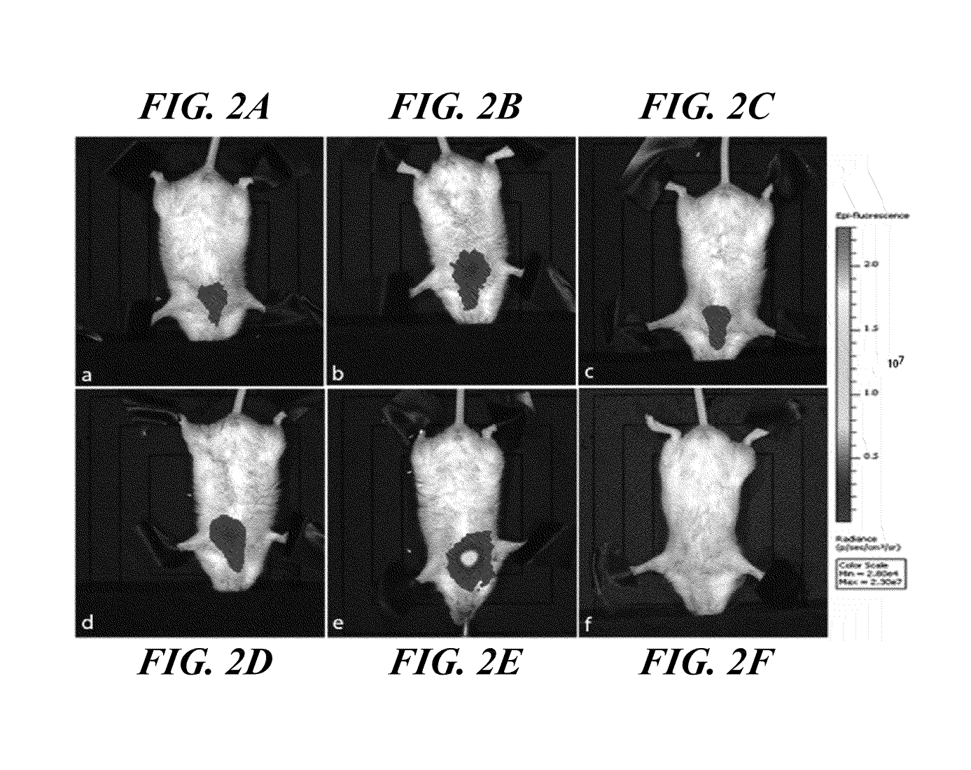

As described above, using the combined localized excitation through the fiber bundle and detection system of the IVIS imaging system, whole animal images were acquired in vivo of tdTomato expressing BCG in the mouse lung, as shown in FIGS. 2A-2F. While signal was detectable for all infectious doses ranging from 10¹ to 10⁵ CFU, as compared to the negative control, it should be noted that only one of the mice infected with the 10¹ CFU inoculation displayed signal. This signifies that, regardless of the presence of bacteria in the microendoscope field of view, light propagation inside the lumen of the lungs may allow for excitation of bacterial fluorescence and detection of this signal using the IVIS CCD.

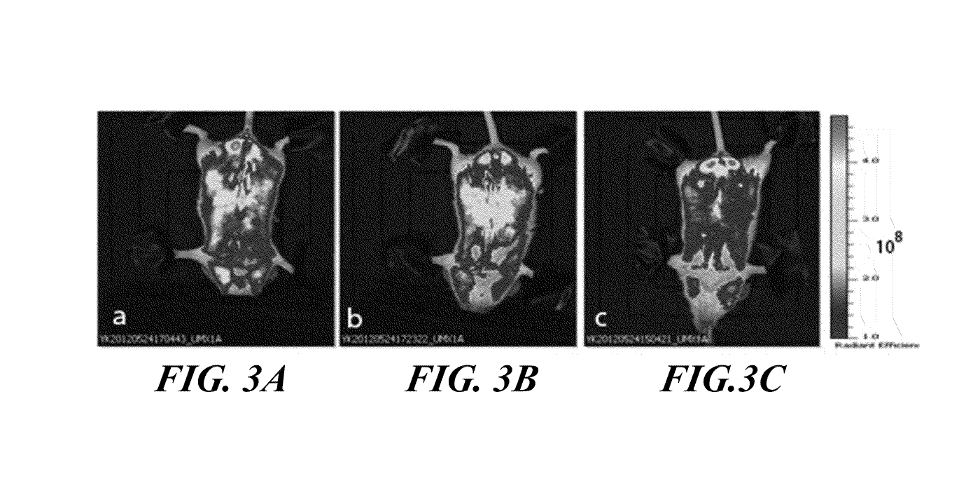

The images acquired with epi-illumination and detection are shown in FIGS. 3A-3C. While fluorescent signal is present in each of these images, fluorescent signal arising from the bacteria is not detectable over the autofluorescence background from the skin and other organs in the animal, such as the gastrointestinal tract and bladder. Conventional epi-illumination of the IVIS imaging system has been unable to detect signal from bacteria in the lung up to 10⁷ CFU of tdTomato bacteria.

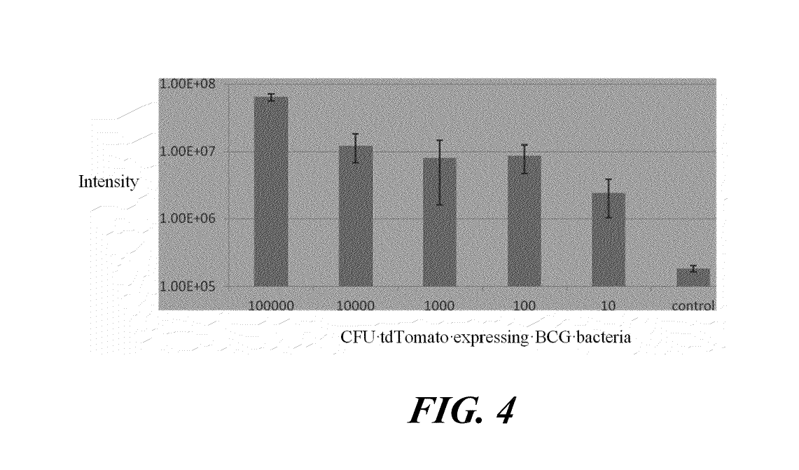

Fluorescence intensities from the IVIS images obtained using localized excitation for each inoculation dose were measured and are plotted in FIG. 4. A Student's t-test reveals 10⁴ CFU to be the lowest inoculum with a statistically significant mean as compared to the negative control (p-value=0.047). The 10⁵ CFU displayed a considerably lower p-value (0.005) as compared to the negative control. Significant variation was seen in the intensities measured for the 10³ CFU inoculation, resulting in a p-value (p=0.19) much greater than the threshold for statistical significance (p<0.05). One cause of this is the variability in infectious dose seen in each group.

By incorporating the microendoscope described herein as a localized excitation source into a whole-animal imaging system, the method of the invention demonstrates the ability to image bacterial infections below the detection limit of traditional epi-illumination whole animal imaging scanners. Bacterial infections were detected with statistical significance (p>0.05) at 10⁴ CFU with three animals per group. With IVIS excitation and detection, bacterial infections at 10⁷ CFU could not be detected in vivo. The method of the invention has the potential to improve detection sensitivity of whole animal imaging systems by reducing losses in excitation intensity due to tissue absorption and scattering. The use of further red-shifted fluorescent proteins could further improve these detection limits. The method exemplified above demonstrates effectiveness in the detection of respiratory infections. However, the method can be applied to areas where whole animal imaging is commonly used, such as gastrointestinal and pancreatic cancer research.

While illustrative embodiments have been illustrated and described, it will be appreciated that various changes can be made therein without departing from the spirit and scope of the invention.

1. A method for detecting fluorescence from a body, comprising:
(a) introducing a light source into a body;
(b) illuminating a portion of the body with the light source, wherein the light source emits light having a wavelength and intensity sufficient to elicit fluorescence from at least a portion of the body illuminated; and
(c) detecting the fluorescence from outside the body.

2. The method of claim 1, wherein detecting the fluorescence outside the body comprises measuring fluorescence externally from the whole body or measuring fluorescence externally from a portion of the whole body.

3. The method of claim 1, wherein the portion of the body illuminated with light comprises a fluorescent agent introduced into the body as a fluorescent agent or as a latent fluorescent agent.

4. The method of claim 3, wherein the fluorescent agent or the latent fluorescent agent is a cell.

5. The method of claim 3, wherein the fluorescent agent or the latent fluorescent agent is a pathogen.

6. The method of claim 1, wherein the light is transmitted through a light guide.

7. The method of claim 1, wherein the light is transmitted through an optical fiber or an optical fiber bundle.

8. The method of claim 1, wherein the light source is a light-emitting diode, a laser diode, a laser, or an incoherent light source.

9. The method of claim 1, wherein introducing the light source into the body comprises introducing the light source through an external orifice of the body.

10. The method of claim 9, wherein the orifice is selected from the oral, nasal, anal, and vaginal orifices.

11. The method of claim 1, wherein introducing the light source into the body comprises introducing the light source through an incision.

12. The method of claim 1, wherein introducing the light source into the body comprises introducing the light source with a catheter.

13. The method of claim 1, wherein the excitation wavelength is one or more wavelengths of visible or near-infrared light.

14. The method of claim 1, wherein detecting the fluorescence comprises measuring the fluorescence using a photodiode, a photodiode array, a photomultiplier tube, a CCD camera, or CMOS camera.

15. The method of claim 1, wherein detecting the fluorescence comprises whole body imaging.

16. A method for monitoring the progression of a disease or condition, comprising:
(a) introducing a light source into a body of a subject;
(b) illuminating a portion of the body with the light source, wherein the light source emits light having a wavelength and intensity sufficient to elicit fluorescence from at least a portion of the body illuminated, and wherein the portion of the body illuminated comprises a fluorescent substance indicative of the state of a disease or condition;
(c) detecting the fluorescence from outside the body to provide a first fluorescent measurement;
(d) waiting a pre-determined period of time;
(e) illuminating the portion of the body as in step (b);
(f) detecting the fluorescence from outside the body to provide a second fluorescent measurement; and
(g) comparing the first fluorescent measurement to the second fluorescent measurement to determine the progression of the disease or condition.

17. The method of claim 16 further comprising repeating steps (d)-(g).

18. The method of claim 16, wherein the fluorescent substance is a cell.

19. The method of claim 16, wherein the fluorescent substance is a pathogen.

20. A method for monitoring the effectiveness of a therapeutic agent, comprising:
(a) introducing a light source into a body of a subject;
(b) illuminating a portion of the body with the light source, wherein the light source emits light having a wavelength and intensity sufficient to elicit fluorescence from at least a portion of the body illuminated, and wherein the portion of the body illuminated comprises a fluorescent substance indicative of the state of a disease or condition;
(c) detecting the fluorescence from outside the body to provide a first fluorescent measurement;
(d) administering a therapeutic agent to the subject;
(e) illuminating the portion of the body as in step (b);
(f) detecting the fluorescence from outside the body to provide a second fluorescent measurement; and
(g) comparing the first fluorescent measurement to the second fluorescent measurement to determine the effectiveness of the therapeutic agent.

21. The method of claim 20 further comprising repeating steps (e)-(g) to provide a profile.

22. The method of claim 20 further comprising repeating steps (d)-(g) to provide a profile.

23. The method of claim 20, wherein the fluorescent substance is a cell.

24. The method of claim 20, wherein the fluorescent substance is a pathogen.
